(19) United States
(12) Patent Application Publication
(10) Pub. No.: US 2005/0079495 A1
(43) Pub. Date: Apr. 14, 2005

(54) Modified tachykinin receptors

(76) Inventors: Matthew Duncan Hall, Cambridge (GB); Shaun McNulty, Cambridge (GB); James Hamer Murray, Bath (GB); Nirmala Suman-Chauhan, Cambridgeshire (GB)

Correspondence Address: David R Kurlandsky;Warner Lambert Company, 2800 Plymouth Road, Ann Arbor, MI, 48105, US

(21) Appl. No.: 10/451,304

(22) PCT Filed: Dec. 5, 2001
(86) PCT No.: PCT/GB01/05404

(30) Foreign Application Priority Data
Dec. 19, 2000 (GB) 0031148.0

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The invention provides a modified tachykinin receptor in which the three amino acids of the DRY sequence that occurs adjacent to the junction of the TM3 domain with intracellular loop 2 are replaced with amino acids whose side chains are neither lipophilic nor contain charged groups. The receptor exhibits similar ligand binding characteristics to the wild type receptor but is incapable or substantially incapable of initiating an endogenous signal. Thus the ligand exhibits no or substantially no intra-cellular coupling of the receptor to the G protein, whereby there is substantially no transduction of ligand binding signals to the cell, The invention also includes fragments of the receptor containing the modified DRY sequence, and polynucleotides that encode the modified receptor as aforesaid. Therapeutic and diagnostic uses for the receptor are disclosed.

### FIELD OF THE INVENTION

The present invention relates to a nucleic acid sequence encoding a tachykinin receptor, the tachykinin receptor encoded by said sequence, methods for its preparation and its use in therapy and screening. In particular, the invention relates to a modification to tachykinin receptor proteins that gives rise to unexpected and useful properties.

### BACKGROUND TO THE INVENTION

Tachykinins are important in the mediation of many physiological and pathological processes including inflammation, pain, migraine, headache and allergy induced asthma. They belong to an evolutionary conserved family of peptide neurotransmitters that have an established role in neurotransmission. They share the C-terminal sequence Phe-Xaa-Gly-Leu-Met-NH₂ (SEQ ID NO 12) in which Xaa represents a hydrophobic residue. That sequence is characteristic of tachykinins and believed to be mainly responsible for their biological activity at neurokinin receptors.

Mammalian tachykinins include substance P (SP), neurokinin A (NKA) and neurokinin B (NKB) which exert their effects by binding to specific receptors. SP is the most prominent member of the tachykinergic system and is released from sensory nerve endings throughout the body. Its amino acid sequence is:
H-Arg-Pro-Lys-Pro-Gln-Gln-Phe-Phe-Gly-Leu-Met-NH₂ (SEQ ID N^{o}1)
The amino acid sequence for NKA is:
H-His-Lys-Thr-Asp-Ser-Phe-Val-Gly-Leu-Met-NH₂ (SEQ ID N^{o}2)
The amino acid sequence for NKB is:
H-Asp-Met-His-Asp-Phe-Phe-Val-Gly-Leu-Met-NH₂ (SEQ ID N^{o}3)

SP has been implicated in the pathology of numerous diseases. For example, it has been shown to be involved in the transmission of pain, in conditions associated with vasodilation, smooth muscle contraction, bronchoconstriction, activation of the immune system and neurogenic inflammation. It has been implicated in migraine, as well as in disorders of the central nervous system, such as anxiety and schizophrenia; in respiratory and inflammatory diseases, such as asthma and rheumatoid arthritis; and in gastrointestinal (GI) disorders and diseases of the GI tract, such as ulcerative colitis and Crohn's disease.

Tachykinin receptors include NK-1, NK-2 and NK-3 and are membrane proteins of the super-family of guanine nucleotide-binding protein (G-protein)-coupled receptors (GPCR). They have extra-cellular binding sites that have preferential affinities for the ligands SP, NKA and NKB respectively. Like other G-protein coupled receptors, they possess an extra-cellular N-terminus, an intra-cellular C-terminus and seven trans-membrane (TM) α-helices of 20-30 amino acids connected by first, second and third extra-cellular loops and by first, second and third intra-cellular or cytoplasmic loops. The greatest homology is found in the membrane-spanning α-helices of trans-membrane domains TM1-TM7 while the N- and C-termini show greater diversity between the three types of neurokinin receptor.

Cloned NK-1 receptors have been reported, including those for the Rana catesbeina, (Simmons et al., Neuroscience, 79: 1219-1229 (1997)), Mus musculus, (Sundelin et al, Eur. J. Biochem. 203: 625-631 (1992)), Rattus norvegicus, (Hershey et al, J. Biol Chem., 266: 4366-4374 (1991) and Yokota et al, J. Biol. Chem., 264: 17649-17652 (1989)), Cavia porcellus, (Gorbulev et al., Biochem. Biophys. Acta 1131: 99-102 (1992)), and Homo sapiens (human), (Takeda et al, Biochem. Biophys. Res. Comm. 179: 1232-1240 (1991) and Fong et al U.S. Pat. No. 5,525,712 and U.S. Pat. No. 5,584,886). Cloned rat and bovine neurokinin-2 receptors have been reported (Y. Sasi et al., Biochem. Biophys. Res. Comm., 165: 695 (1989), and Y. Masu, et al., Nature 329: 836 (1987)). Cloned rat neurokinin-3 receptor has also been reported (R. Shigemoto, et al., J. Biol. Chem., 265:623 (1990)).

All three receptors share the signal transduction mechanisms of a G-protein coupled receptor. The receptor is in an OFF state when no ligand is present, but, is triggered into an ON state when an agonist ligand binds to the receptor. The G-protein when in its ON state triggers a downstream signaling pathway or signal cascade. The G-protein comprises α-, β- and γ-units that are bound together in the OFF state, the α-subunit then having GDP bound to it. In the ON state GTP replaces the GDP bound to the α-subunit. The α-subunit becomes dissociated from the β- and γ-subunits and becomes available for activating the signal cascade. After a short period, the GTP becomes hydrolyzed to GDP and the G-protein returns to its non activated OFF state. Hydrolysis provides a negative feedback mechanism that ensures that the G protein is only in its activated ON state for a short period.

Various studies have been undertaken, involving different G-protein receptors, to determine how the various regions of the protein structure affect intra-cellular coupling of the receptor to the G-protein and consequential transduction of ligand binding signals to the cell. G-protein receptors have a well-conserved sequence in the second intracellular loop where the loop joins the third trans-membrane domain that is known as the DRY sequence. It has the residues
5′-DRYXXV(P)XXPL-3′ (SEQ ID N^{o}4)
in which L represents Leu, Ile, Val, Met or Phe and X represents any amino acid. It has been suggested that the DRY sequence contributes to the efficient binding and activation of G-proteins. Fraser et al., Proc. Natl Acad Sci. USA, 85: 5478-5482 (1988) report a change of Asp to Asn at position 130 of the human β-adrenergic receptor (i.e. the D of the DRY sequence) resulting in human β-adrenergic receptor that exhibits high affinity binding of agonist whilst being unable to interact effectively with G-protein. However, a second paper from the same laboratory reports that the previous very high agonist binding efficiency in human β-adrenergic receptor mutated at position 130, upon which the above mentioned conclusion had been based, had not been reproduced (Wang et al., Mol Pharmacol 40(2): 168-79 (1991)). In a review article Savarese and Fraser said that this locus may be important for coupling to some, but not all, G-proteins (Biochem J., 283: 1-19 (1992)). Moro et al made mutants of the Hm1 muscarinic cholinergic receptor with changes towards the 5′-end of the DRY sequence and found that replacing L at position 131 with A gave the strongest reduction in coupling efficiency (J. Biol. Chem. 268: 22273-22278 (1993)). Subsequently, Shibata et al made a mutant of the angiotensin II receptor type I in which DRY at positions 125-127 is replaced by GGA and M at position 134 is replaced by A, resulting in uncoupling of the mutant A receptor from G-proteins (Biochem. Biophys. Res. Com. 218: 383-389 (1996)). The authors concluded that DRY sequence as a whole including the final lipophilic amino acid L serves as a general site for G-protein coupling but they did not go on to consider what effects might be obtained by change confined to the DRY portion of the sequence.

Comparing the binding affinities of the two known isoforms of the human NK-1 receptor shows the importance of the cytoplasmic tail. The long form (407 amino acids) and short form (311 amino acids) differ in the length of the C-terminus. The long form has similar substance P binding characteristics to the rat NK-1 receptor, while the short form of the receptor has an apparent substance P binding affinity 10-fold less than the rat NK-1 receptor.

Furthermore, studies on these and other receptors have shown that the effect of mutations is unpredictable and specific to certain families of receptors. Hence, a change in one G-protein coupling family will not necessarily have the same effect on another. Other attempts to distinguish protein binding and signaling effects bear this out.

### SUMMARY OF THE INVENTION

The invention provides a mutant tachykinin receptor in which the three amino acids of the DRY sequence that occurs adjacent to the junction of the TM3 domain with intracellular loop 2 are replaced with amino acids whose side chains are neither lipophilic nor contain charged groups, said receptor exhibiting similar ligand binding characteristics to the wild type receptor but being incapable or substantially incapable of initiating an endogenous signal. Thus the ligand exhibits no or substantially no intra-cellular coupling of the receptor to the G-protein, whereby there is substantially no transduction of ligand binding signals to the cell. The way in which tachykinin receptors attach to cell membranes, the trans-membrane domains extra-cellular and cytoplasmic loops and the place where the DRY sequence referred to above occurs are apparent by inspection of FIG. 5 of the accompanying drawings.

The invention further provides any of the following:
a fragment of said mutant tachykinin receptor containing said modified DRY sequence;
an isolated protein or polypeptide containing an amino acid sequence at least 95% identical to the above sequence;
a variant thereof with sequential amino acid deletions from either the C terminus or the N-terminus; and
an allelic variant, heterospecific homologue or biologically active proteolytic or other fragment thereof containing said modified DRY sequence.

The invention also comprises an isolated cell membrane in which a modified tachykinin receptor as aforesaid is incorporated as membrane protein. Such cell membrane material finds utility for research and in particular for screening for therapeutically useful compounds as described below.

The invention yet further provides any of the following:
(a) an isolated nucleic acid molecule comprising a polynucleotide that encodes a modified tachykinin receptor as aforesaid;
(b) an isolated nucleic acid molecule comprising a sequence that is hybridizable to the above sequence;
(c) a gene which is the result of extending the above sequence or any sequence that is hybridizable to the above sequence;
(d) a sequence or gene that is functionally equivalent to the above sequence or to a gene that is an extension of the above sequence, i.e. that is not identical to the sequence or gene referred to but functions biologically as equivalent to the sequence or gene referred to, including any allelic variants and heterospecific mammalian homologues, including artificial or recombinant sequences created from cDNA or genomic DNA;
(e) a recombinant vector comprising the above gene sequence; and
(f) a host cell transformed with the vector.

The invention also provides a method for producing one of the amino acid sequences described herein and especially the receptor protein defined by SEQ ID N^{o}5, which method comprises the steps of:
(a) inserting said nucleic acid sequence into an appropriate vector;
(b) culturing in a culture medium a host cell previously transformed or transfected with the recombinant vector of step (a);
(c) harvesting cells containing the receptor protein obtained from step (b); and
(d) separating or purifying from said culture medium or from said host cells the thus-produced receptor protein. In step (d) of the above method, the receptor protein may be obtained either from the culture medium and/or by lysing the host cell, for example by sonication or osmotic shock.

The invention yet further provides a method for screening for therapeutically active compounds, said method comprising the following steps:
(a) providing a cell line expressing a modified tachykinin receptor as aforesaid;
(b) adding test sample to a solution containing labeled SP or other tachykinin ligand and the cell line from step (a);
(c) incubating the cell line, test sample and labeled SP or other ligand mixture from step (b) to allow binding of SP or other ligand and test sample to the modified tachykinin receptor;
(d) optionally separating the non-bound labeled SP or other ligand from the labeled SP or other ligand bound to the modified tachykinin receptor and, if desired,
(e) measuring the amount of labeled SP or other ligand that is bound to the modified tachykinin receptor.

The invention further provides:
(a) the use of a modified tachykinin receptor according to the invention in the preparation of a medicament for the treatment or prophylaxis of a condition associated with substance P or other tachykinin (neurokinin) receptor-binding ligand;
(b) the use of such a modified tachykinin receptor in therapy;
(c) a method for the treatment or prevention of a condition associated with over-expression of an endogenous tachykinin ligand, which method comprises administration to a patient in need thereof of a non-toxic, effective amount of such a modified tackykinin ligand as described above;
(d) a composition comprising a modified tachykinin ligand as described above in association with a pharmaceutically and pharmacologically acceptable carrier therefor; and
(e) a use, method or composition according to any one of (a) to (d) above, in which the modified tachykinin ligand becomes generated in vivo from a nucleic acid sequence encoding such a ligand.

### BRIEF DESCRIPTION OF THE DRAWINGS

The invention will now be further described with reference to the accompanying drawings, in which:

FIG. 1: describes a protein sequence, being a translation of the sequence shown in FIG. 1 and being the sequence of a modified human NK-1 receptor according to the invention (hNK-1Rv1).

FIG. 2: is a consensus cDNA sequence encoding a modified human NK-1 receptor according to the invention.

FIG. 3: is a bar chart showing the results of Calcium imaging experiments on NK-1 wild type receptor and the modified receptor of FIG. 1 (hNK-1Rv1), and wild type receptor antisense transfected COS cells.

FIG. 4: shows the sequence alignments between NK-1 and other NK receptors in various species in the region of the DRY motif in TM3, demonstrating high conservancy (In the Figure, NK-1 sequences 1-4 provide SEQ ID NO 13, NK-1 sequence 5 provides SEQ ID NO 14, NK-2 sequences 1-2 and 4-7 provide SEQ ID NO 15, NK-2 sequence 3 provides SEQ ID NO 16 and NK-3 sequences 1-3 provide SEQ ID NO 17).

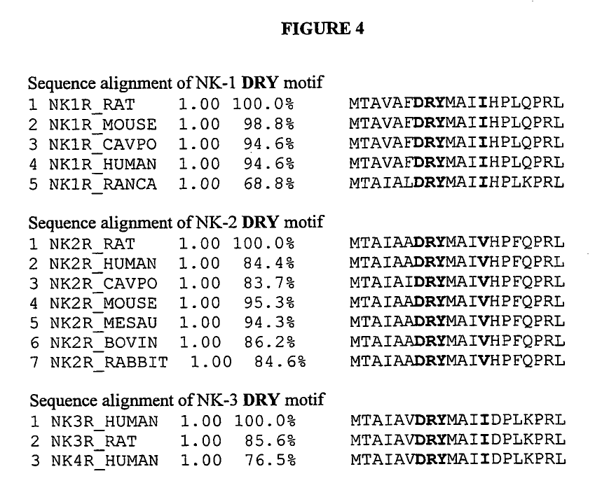

FIG. 5: is a diagram showing the nNK-1Rv1 receptor and portions of the cell membrane into which it is incorporated (SEQ ID NO 18; in addition to the DRY motif, L223 is changed to I; this change is believed to be inconsequential).

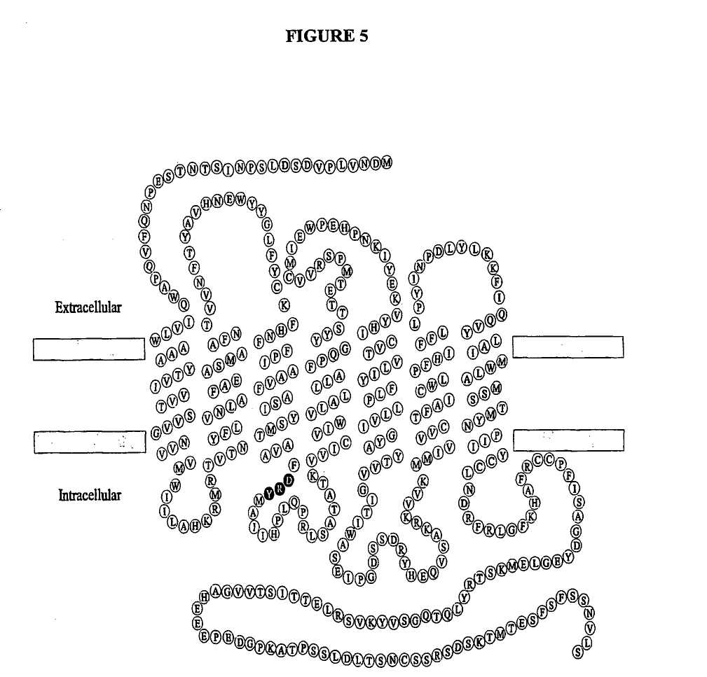

### DESCRIPTION OF PREFERRED EMBODIMENTS

Definitions

Definitions for a number of terms used in this specification are given below:

Alleles or allelic sequences mean alternative forms of the receptor genes referred to above resulting from one or more variations in the nucleic acid sequence, resulting in altered mRNAs and proteins or polypeptides

Functional equivalence when used in relation to gene sequences or amino acid sequences covers sequences that are not identical to the sequence referred to but function biologically or chemically as equivalents of the disclosed sequence.

Incapable of initiating an endogenous signal in the present context means that, within experimental error (±5% of control), the modified NKR does not result in conversion of GTP to GDP in the cell or a recombinant construct; and/or
does not evoke a calcium flux therein; and/or
does not evoke other components of the signal transduction pathway.

Isolated, when used in relation to a polynucleotide sequence, means such a sequence that has been removed from its natural environment, i.e. from the organism in which it occurs in nature and/or from genes that are immediately contiguous (one at the 5′ end and the other at the 3′ end) in the naturally occurring genome of the organism from which it is derived. Isolated when used in relation to a cell membrane refers to the membrane as a discrete entity substantially separate from cytoplasmic material.

Operably linked refers to a linkage of polynucleotide elements in a functional relationship. For instance, a promoter or an enhancer is operably linked to a coding sequence if it affects the transcription of the coding sequence. More specifically, two DNA molecules (such as a polynucleotide containing a promoter region and a polynucleotide encoding a desired polypeptide or polynucleotide) are said to be “operably linked” if the nature of the linkage between the two polynucleotides neither results in the introduction of a frame-shift mutation nor interferes with the ability of the polynucleotide containing the promoter to direct the transcription of the coding polynucleotide.

Stringent hybridisation conditions is a recognized term in the art and for a given nucleic acid sequence refers to those conditions which permit hybridisation of that sequence to its complementary sequence and not to a substantially different sequence. It generally implies at least about 97% identity between the sequences.

Protein and Nucleic Acid Sequences

In the above mentioned mutant receptors, amino acids for modifying the DRY sequence that are non-polar and have the correct hydrophilic/lipophilic balance include glycine and alanine which are preferred. However, the inventors also envisage the use of other non polar amino acids.

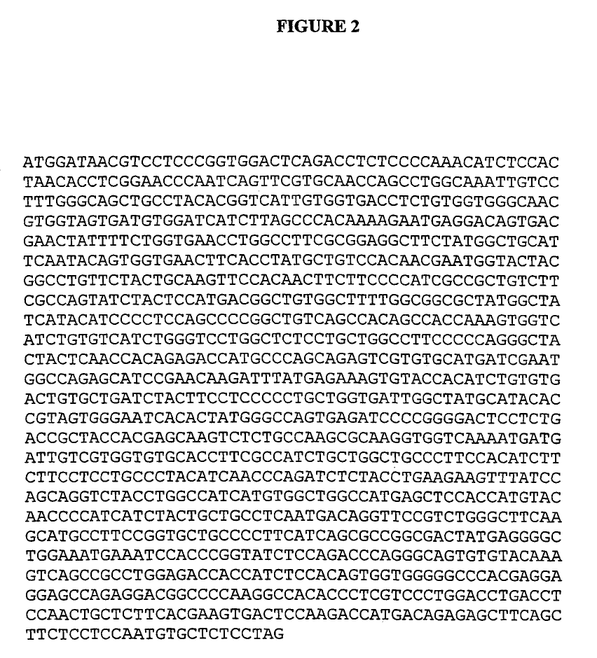

In a preferred aspect, the invention provides a nucleic acid sequence as shown in FIG. 2 [SEQ ID N^{o}6], which encodes the consensus amino acid sequence for the modified hNK-1v or a sequence that hybridizes thereto under stringent hybridization conditions.

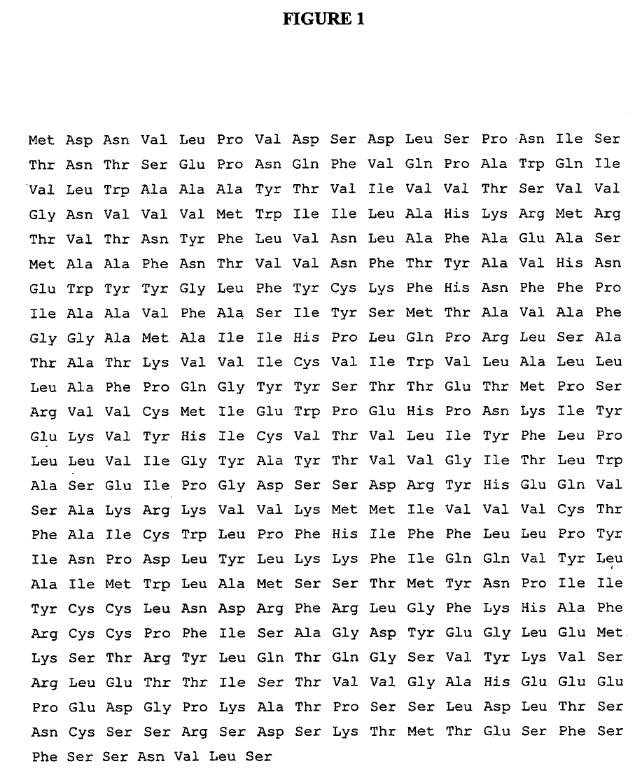

The invention also provides a preferred hNK-1v receptor sequence shown in FIG. 1 [SEQ ID N^{o}5].

The invention further provides a nucleic acid sequence encoding a receptor protein having at least 80%, preferably 90%, more preferably 95%, and most preferably 98% amino acid identity with the hNK-1Rv1 encoded by the nucleic acid sequence of SEQ ID N^{o}6, and which
encodes protein that can bind to SP, but cannot initiate its endogenous signal, or
encodes a peptide fragment thereof according to sequence (d), or
encodes a sequence complementary thereto according to sequence (e), as defined hereinabove.

The invention also provides a protein that:
(a) has at least 80%, preferably 90%, more preferably 95%, and most preferably 98% amino acid identity with the hNK-1Rv1 protein having the amino acid sequence of SEQ ID N^{o}5; and
(b) can bind to SP or to a peptide fragment thereof, or to a sequence complementary thereto, but cannot initiate its endogenous signal to G protein.

The hNK-1Rv1 variant of the NK-1 receptor shown in SEQ ID N^{o}5 has similar ligand binding characteristics to the wild type receptor, but is deficient in cell signaling capabilities, as demonstrated by the results of tests described in Examples 4 and 5. Some mammalian receptors may exhibit overall homology with the hNK-1 receptor of less than 80% (for example, rat NK-2 receptor has about 48% homology to rat NK-1 receptor), but are nevertheless included within the scope of this invention in view of their conservancy in the intracellular DRY region of TM3. Accordingly, the present invention provides a polypeptide having as low as 40% overall amino acid identity with the hNK-1Rv1 protein, but having at least 75%, such as at least 80%, preferably 90%, more preferably 95%, and most preferably 98% amino acid identity with the TM3 intracellular loop of the bNK-1Rv1 protein in the vicinity of the DRY motif, and having the properties previously specified.

In view of the high level of conservancy demonstrated by other NK receptors in the DRY region of TM3 intracellular loop, and also the high level of conservancy observed in that region between human and other species, the present invention also provides a nucleotide sequence that encodes a modified neurokinin receptor, wherein the modification is or includes substitution of the DRY motif within the intracellular loop of TM3, namely, Asp¹²⁹, Arg¹³⁰ and Tyr¹³¹, by Gly, Gly and Ala, respectively, for example wherein the receptor is a modified NK-2 or modified NK-3 mammalian receptor.

Vectors

The invention further provides a vector (e.g. a plasmid or virus) comprising a nucleic acid encoding the modified tachykinin receptor, especially the hNK-1Rv1 receptor or any other modified hNK-1R of this invention.

A recombinant vector of the invention comprises an expression vector comprising a nucleic acid sequence encoding the modified NK-R, especially the hNK-1Rv1, polypeptide. One suitable vector for the expression of a human variant NK-1 receptor of the invention is a baculovirus vector that can be propagated in insect cells and in insect cell-lines. Expression requires that appropriate signals are provided in the vector, said signals including various regulatory elements such as enhancers/promoters from both viral and mammalian sources that drive expression of the genes of interest in host cells. The regulatory sequences of the expression vectors are operably linked to the nucleic acid encoding the modified tachykinin receptor, especially the human NK-1 variant receptor. Generally, recombinant expression vectors include origins of replication, selectable markers permitting transformation of the host cell, and a promoter derived from a highly expressed gene to direct transcription of a downstream structural sequence. The heterologous structural sequence is assembled in an appropriate frame with the translation, initiation and termination sequences, and preferably a leader sequence capable of directing sequences of the translated protein into the periplasmic space or the extra-cellular medium. Where the vector is adapted for transfecting and expressing desired sequences in eukaryotic host cells, preferred vectors comprise an origin of replication from the desired host, a suitable promoter and an enhancer, and also any necessary ribosome binding sites, polyadenylation site, transcriptional termination sequences, and optionally 5′-flanking non-transcribed sequences.

Suitable promoter regions used in the expression vectors according to the invention are chosen taking into account the host cell in which the heterologous nucleic acids have to be expressed. A suitable promoter may be heterologous with respect to the nucleic acid for which it controls the expression, or may be endogenous to the native polynucleotide containing the coding sequence to be expressed. Additionally, the promoter is generally heterologous with respect to the recombinant vector sequences within which the construct promoter/coding sequence has been inserted.

A recombinant vector of the invention may be used to amplify a polynucleotide derived from the nucleic acid sequence encoding the modified NKR, especially the hNK-1Rv1 polypeptide that has been inserted in a suitable host cell, this polynucleotide being amplified every time the recombinant vector replicates.

DNA sequences derived from the SV40 viral genome, for example SV40 origin, early promoter, enhancer, and polyadenylation sites may be used to provide the require non-transcribed genetic elements.

The suitable promoter regions used in the expression vectors according to the invention are chosen taking into account the host cell in which the heterologous nucleic acids are to be expressed. A suitable promoter may be heterologous with respect to the nucleic acid for which it controls the expression or alternatively it can be endogenous to the native polynucleotide containing the coding sequence to be expressed.

Additionally, the promoter is generally heterologous with respect to the recombinant vector sequences within which the construct promoter/coding sequence has been inserted. Preferred bacterial promoters are the LacI, LacZ, T3 or T7 bacteriophage RNA polymerase promoters, the lambda PR, PL and trp promoters (EP 0 036 776), the polyhedrin promotor, or the p10 protein promoter from baculovirus (Kit Novagen; Smith et al., 1983); O'Reilly et al., 1992, Baculovirus expression vectors: A Laboratory Manual. W.H. Freeman and Co., New York).

Preferred selectable marker genes contained in the expression recombinant vectors of the invention for selection of transformed host cells are preferably dihydrofolate reductase or neomycin resistance for eukaryotic cell cultures, TRP1 for S. cerevisiae or tetracyclin, rifampicin or ampicillin resistance in E. coli, or Levan saccharase for mycobacteria, this latter marker being a negative selection marker.

Preferred bacterial vectors of the invention are listed hereafter as illustrative but not limitative examples:
pQE70, pQE60, pQE-9 (Qiagen), pD10, fephagescript, psiX174, p.Bluescript SK, pNH8A, pNH16A, pNH18A, pNH46A (Stratagene); pKK223-3, pKK233-3, pDR540, pRIT5 (Pharmacia); pWLNEO, pSV2CAT, pOG44, pXT1, pSG (Stratagene); pSVK3, pBPV, pMSG, pSVL (Pharmacia); pQE-30 (Qiagen).

Preferred bacteriophage recombinant vectors of the invention are P1 bacteriophage vectors such as described by Sternberg N. L. (1992;1994).

A suitable vector for the expression of hNK-1vRa polypeptide of the invention or a fragment thereof, is a baculovirus vector that can be propagated in insect cells and in insect cell-lines. A specific suitable host vector system is the pVL 1392/1393 baculovirus transfer vector (Pharmingen) that is used to transfect the SF9 cell line (ATCC N^{o}CRL 1711) which is derived from spodoptera frugiperda.

The recombinant expression vectors of the invention may also be derived from an adenovirus such as those described by Feldman and Steg. (1996) or Ohno et al. (1994).

Another preferred recombinant adenovirus according to this specific embodiment of the present invention is the human adenovirus type two or five (Ad 2 or Ad 5) or an adenovirus of animal origin (French Patent Application n^{o}FR 93 05 954).

Particularly preferred retroviruses for the preparation or construction of retroviral in vitro or in vivo gene delivery vehicles of the present invention include retroviruses selected from the group consisting of Mink-Cell Focus Inducing Virus, murine sarcoma virus, and Ross Sarcoma Virus. Other preferred retroviral 5 vectors are those described in Roth et al. (1996), in PCT Application WO 93/25 234, in PCT Application WO 94/06920, and also in Roux et al. (1989), Julan et al. (1992) and Nada et al. (1991).

Yet, another viral vector system that is contemplated by the invention consists in the adeno associated virus (AAV) such as those described by Flotte et al. (1992), Samulski et al. (1989) and McLaughlin et al. (1996).

Expression Systems

For expression of the modified neurokinin receptors, the invention provides host cells transformed (prokaryotic cells) or transfected (eukaryotic cells) with such a vector; and any cell, or live organism, including a non-human mammal, that has been genetically engineered to produce such a polypeptide, said cell or live organism incorporating expressibly therein a nucleic acid sequence according to this invention.

Heterologous expression systems may be used to express cloned NK-1 receptor, NK-2 receptor and NK-3 receptor, including human and non-human mammalian variants thereof. The choice of expression system depends on a number of factors including stability of protein expression, post translational modification and required yield. However, as a general rule, the more complex the organism the lower the yield of expressed receptor, but the greater the likelihood that the receptor will be in its native conformation. Several expression hosts are commonly available

Prokaryotic host cells, e.g. Escherichia coli DH5-α (see Sambrook et al., for a comprehensive guide to gene expression in E. coli).
Yeasts, e.g. Pichia pastoris. The feasibility of expressing NK-1 receptor in yeast has been demonstrated by Arkinstall et al (1995, FEBS 275 183-187) who successfully expressed therein the closely related receptor, human NK-2. Large-scale production of a human G-protein coupled receptor in yeast was demonstrated by Sizemann et al (1996, Receptors and Channels, Vol 4, 197-203). In brief, a protocol to express a functional NK-1 receptor in yeast can be represented by: (1) splicing the NK-1 cDNA into a yeast expression vector; (2) Transforming this vector into yeast; and (3) Selecting for yeast containing the NK-1 cDNA and expression of the NK-1 cDNA.
Eukaryotic host cells, e.g. insect cells, non-mammalian vertebrate cells and mammalian cells. Non-mammalian vertebrate cell lines include Xenopus (frog) oocytes. Other cell lines which are contemplated in this invention include HeLa cells (ATCC N^{o}CCL2; N^{o}CCL2.1; N^{o}CCL2.2), Cv 1 cells (ATCC N^{o}CCL70), COS cells (ATCC N^{o}CRL 1650; N^{o}CRL 1651), Sf-9 cells (ATCC N^{o}CRL 1711), C127 cells (ATCC N^{o}CRL-1804), 3T3 cells (ATCC N^{o}CRL-6361), CHO cells (ATCC N^{o}CCL-61), human kidney 293 cells (ATCC N^{o} 45504; N^{o}CRL-1573) and BHK (ECACC N^{o}84100 501; N^{o}84111301); PC12 (ATCC N^{o} CRL-1721), NT2, SHSY5Y (ATCC N^{o} CRL-2266), NG108 (ECACC N^{o}88112302) and F11, SK—N—SH (ATCC N^{o} CRL-HTB-11), SK—N—BE(2) (ATCC N^{o} CRL-2271), IMR-32 (ATCC N^{o} CCL-127). A preferred system to which the gene of the invention can be expressed are cell lines such as COS cells, 3T3 cells, HeLa cells, 292 cells and CHO cells. A preferred system for the efficient expression of hNK-1vR involves the use of CHO and COS cell lines. The gene can be expressed through an endogenous promoter of native CHO or COS, or through an exogenous promoter. Suitable exogenous promoters include such as SV40 and CMV, or perhaps a eucaryotic promoter such as the tetracycline promoter. The preferred promoter being CMV.

In some instances, it may be required to tag e.g. a human NK-1 variant receptor prior to purification. The tag is then, in most instances, encoded into the nucleotide sequence that is needed to express the polypeptide. Examples of such tags include, but are not limited to sequences encoding C-myc, FLAG, a sequence of histidine residues, haemaglutin A, V5, Xpress or GST. Most of these tags can be incorporated directly into the sequence, for instance through PCR amplification by incorporating the appropriate coding sequence in one of the PCR amplification primers. However, the tag can also be introduced by other means, such as by covalent binding of the appropriate nucleic acid sequence encoding the tag moiety, such as GST, with the 3′ or 5′ end of the nucleic acid sequence encoding the polypeptide sequence. Purification of the NK variant receptor may, in the case of the use of a histidine tag, then be carried out by passage onto a nickel or copper affinity chromatography column, such as a Ni NTA column. The polypeptide thus produced may optionally be further characterized, for example by binding onto an immuno-affinity chromatography column on which polyclonal or monoclonal antibodies directed to the NK variant receptor have been previously immobilised.

For some purposes it may be useful to provide mammals e.g. mice, rats or guinea-pigs in which in which modified human or non-human mammalian tackykinin receptors, e.g. NK-1v receptors, are present. Transgenic rats, mice and other mammalian cells may be produced by generating a targeting vector and transfecting the cells to be cultured e.g. using the gene targeting services of, e.g. DNX Transgenic Sciences. In the case of transgenic mammals, stem cells are transfected, cultured to blastocytes and introduced into the uterus of a female mammal. Because they have in their cell membranes tachykinin receptors that bind to endogenous ligands such as SP but are incapable, or substantially incapable, of initiating their endogenous signal, the animals are useful, in research into the effects of these ligands and the developments of transduction inhibitors, especially because they can indicate the behaviour of the animal the presence of endogenous ligand but in the absence of a transduced signal and in the absence of antagonist, and can therefore provide a true control. (Silver, Lee M. Title: Mouse genetics—concepts and applications Publisher: New York; Oxford University Press 1995); P De-Felipe-C et al;Nature. 1998 Mar. 26; 392(6674): 394-7).

Assays

The present invention further provides a modified tachykinin receptor, such as a human NK-1v receptor, used as a substitute in an assay to identify and evaluate entities that bind to the wild type tachykinin receptor.

The invention also includes human NK-1v receptor used as a substitute in an assay in order to determine the concentration of substance P in body fluids in patients with arthritis, pain, migraine, anxiety, schizophrenia, asthma, rheumatoid arthritis, and in gastrointestinal disorders and diseases of the GI tract, like ulcerative colitis and Crohn's disease.

As previously mentioned three tachykinin receptors have been identified, referred to as NK-1, NK-2 and NK-3 and they have respective endogenous lignads SP, NKA and NKB. Although each tachykinin has a preferred ligand, each receptor has the ability to interact with the other tachykinin ligands, and the pathology of cross-ligand binding in disease states is poorly understood. Study of a system where NK-1, NK-2 or NK-3 receptor response either singularly or in tandem has been diminished in vitro or in vivo could significantly aid understanding of the role of tachykinin receptors and their ligands in disease states.

Screening Methods

The invention also provides a method for screening for therapeutically active compounds, said method comprising the following steps:
(a) providing a cell line expressing the modified tachykinin receptor, e.g. the human NK-1v receptor;
(b) adding test sample to a solution containing labeled SP or other ligand and the cell line from step (a);
(c) incubating the cell line, test sample and labeled SP or other ligand mixture from step (b) to allow binding of SP or other ligand and test sample to the modified tachykinin receptor;
(d) optionally separate the non-bound labeled SP or other ligand from the labeled SP or other ligand bound to the modified tachykinin receptor; and, if desired,
(e) measuring the amount of labeled SP or other ligand that is bound to the modified tachykinin receptor.

Preferably, the assay involves COS-7 cell lines. Cell membranes containing the modified tachykinin receptor, e.g. the human NK-1v receptor, can be used instead of whole cells.

The SP or other tachykinin ligand e.g. NKA or NKB may be labeled by any method known in the screening art, e.g. by a radioactive label, such as ¹²⁵I, or by a fluorescent label. In certain circumstances, (e.g. fluorescence polarization assays), bound and unbound SP or other tachykinin ligand do not have to be separated to quantify the amount of SP or other ligand bound to the receptor. Alternatively, the SP or other ligand may be bound to a matrix, and labelled cells may be used to quantify the binding of a modified tachykinin receptor, such as human NK-1 receptor, to the SP. In this case, the assay procedure may follow the steps set out below:
(a) a cell line is provided that expresses a modified tachykinin receptor, such as the human NK-1v receptor;
(b) the cell line is labeled;
(c) the test sample and labeled cells are added to a matrix binding SP or other ligand;
(d) the labeled cells, test sample and matrix-bound SP or other ligand are incubated to allow binding of SP or other ligand and test sample to the expressed modified tachykinin receptor;
(e) the labeled non-bound cells are separated from the SP or other ligand bound cells; and, if desired,
(f) the amount of labeled cells containing the modified tachykinin receptor, such as the human NK-1v receptor, that has bound to SP or other ligand is measured.

In order to provide transgenic animals or cell lines for use in assays, which animals and/or cell lines comprise a sequence as described herein, general methods are known and may be adapted accordingly. Different types of vectors including modified retroviruses, adenovirus, adeno-associated virus, herpes virus and plasmid DNA have been proposed as vehicles to introduce foreign genetic material into cells or tissues.

Protein Therapy

The invention also encompasses modified tachykinin receptors, especially human NK-1v receptor, for use in protein therapy to reduce the effects of, or an excess of endogenous ligand.

Protein therapy can be used for the suppression of the action of SP in interstitial fluid of the lungs. For example, a purified preparation of a modified tachykinin receptor, such as human NK-1v receptor, (eg a liquid or powder carrier formulation) can be directly administered to the airways. Once in the airways, the tachykinin variant receptor can interact and bind to, for example, SP molecules. This has the effect reducing the amount of SP available for the endogenous NK receptor. Likewise, a modified tachykinin receptor, such as the human NK-1v receptor, can be directly introduced to body cavities such as joints and interstitial lung space where SP is present. The variant receptor has the capability to bind SP, therefor reducing the amount of SP available to interact with the wild type receptor and causing down-regulation of the SP cellular response.

The invention provides a modified NK receptor, such as a human NK-1v receptor, for use in removing or suppressing SP in body fluids, e.g. the interstitial fluid of the lungs and fluid in the cavities of joints. A purified preparation of a modified NK receptor, such as human NK-1v receptor, (e.g. a liquid carrier formulation) may be administered directly to a joint. Once in the fluid- filled joint cavity, the NK variant receptor interacts and binds to SP molecules thereby reducing the amount of SP available to activate the endogenous NK receptor.

The hNK-1v receptor can reduce the effect of excess or inappropriately expressed SP in patients with pain associated with migraine, neuralgia, diabetic, peripheral, AIDS-related and chemotherapy-induced neuropathy, and neuropathies of diverse origin; anxiety and anxiety disorders, such as panic disorder, phobias and obsessive-compulsive behavior; schizophrenia; asthma; rheumatoid arthritis; and in gastrointestinal disorders and diseases of the GI tract, for example ulcerative colitis and Crohn's disease. Other conditions or disease states that can be treated, ameliorated or prevented include: psychosomatic and psycho-immunological disorders; attention deficit disorder; pre-menstrual (PMT or PMS) or late luteal phase syndrome; mania or hypomania; aggressive behavior disorders; emesis, including motion sickness, migraine-induced sickness and that arising from chemotherapy; postherpetic neuralgia; depression; inflammation; eating disorders, such as obesity, bulimia nervosa and compulsive eating disorders; cognitive disorders, such as dementia and amnestic disorders; movement disorders, such as dyskinesias, akinetic-rigid syndromes, Gilles de la Tourette syndrome, tremor, or dystonia; schizophrenic disorders; substance abuse disorders; bipolar disorder; sexual dysfunction, including impotence; stress; alteration of circadian rhythmicity; Alzheimer's disease; bladder disorders; hypertension; angina; ischaemia; multiple sclerosis; chronic obstructive lung disease; scleroderma; CNS disorders; and other conditions where excess tachykinin peptides such as SP are involved. Furthermore, the inventors believe that modification to the DRY motif of NK-2 receptor and NK-3 receptor in a similar manner to that described for the NK-1 receptor above, would allow for the production of modified tachykinin receptors which could be used to remove their specific ligands from body fluids.

Several factors need to be taken into account in protein therapies. These include: solublisation, maintenance of activity and stability of the protein, delivery and dose. The first three points are closely related. Proteins are large relative to conventional drugs and their biological activity is dependent on their primary, secondary, tertiary and in some instances quaternary structure being maintained. They often have labile bonds and numerous chemically reactive groups in their side chains. Disruptions of their structure by denaturation or aggregation can lead to loss of activity or increase in immunogenicity. One of the key problems in devising effective formulations for biologically active proteins is to find a formulation that is both stable and biologically active. The route of administration of the protein also plays a significant role in formulation. Microsphere formulations can be used for injection, aid in the maintenance of stability and activity of the protein, and offer the possibility of slow release formulations. Solid large powder formulations are most suitable for use in aerosol and topical treatments.

Putney and Burke (Nature Biotechnology 1998: 153-157) outline various methods for producing microspheres. One such method is the atomization-freezing process. In this encapsulation method, the micronised solid protein is suspended in biodegradable polymers of DL-lactic co-glycolic acid (PLGA) solution that is then atomised using sonication or air-atomisation. This produces droplets that are then frozen in liquid nitrogen. Addition of ethanol at <−40° C., in which both the protein and the PLGA are insoluble, extracts the organic solvent from the micro-spheres. This process is further described in U.S. Pat. No. 5,019,400. An alternative polymer for use in the process is methylene chloride polymer,to encapsulate growth hormone see Johnson et al, Nature Medicine 2: 795-799 (1996). The following method can be used to incorporate modified NKR, such as human NK-1v receptor, post-purification into microspheres:
(a) concentrate the purified, active microsphere modified NKR, such as human NK-1v receptor to >100 mg/ml in the presence of stabilisers;
(b) add PLGA or methylene chloride polymer solution and mix; then
(c) atomise the frozen suspension in liquid nitrogen to fix the microspheres and extract with ethanol. Microspheres thus produced should have a diameter in the μM order.

The invention further provides a method for treatment of a patient in need thereof, which comprises administering to said patient a composition in the form of an aerosol that comprises a modified tachykinin receptor as described above, e.g. an hNK-1Rv receptor.

SP or other ligand such as NKA or NKB can also be removed directly from biological fluids using the modified tachykinin receptors described above. For example, purified human NK-1v receptor could be bound to a suitable matrix. Biological fluids containing SP can be passed over the matrix, so that the SP preferentially binds to matrix while other components of the fluid do not and SP becomes preferentially removed from the biological fluid.

Nucleic Acid Therapy

The invention further provides a method for gene therapy treatment of a patient in need thereof, which comprises administering to said patient a nucleic acid sequence, virus or plasmid encoding a modified tachykinin receptor as described above, e.g. an hNK-1Rv receptor. Different types of vector including modified retroviruses, adenovirus, adeno-associated virus, herpes virus and plasmid DNA have been proposed as vehicles for introducing foreign genetic material into the cells or tissues of patients, and can in principle be used to introduce the modified tachykinin receptor nucleic acid sequences referred to above. The appropriate strategy for administering the nucleic acid sequence depends on the target tissue, disease state and longevity of the proposed therapy.

Furthermore, down regulation of the effect SP on a cellular system could be achieved by expressing the hNK-1v receptor in cells other than those expressing native NK-1. The expression of SP in these cells (which are in the proximity of cells expressing native NK-1 receptor) could have the effect of “moping” up SP and reducing the available SP for the native receptor to interact with.

Introduction of a modified NK receptor, such as a human NK-1v receptor, (by way of direct introduction of protein or introduction of an expressible gene encoding this protein) into the outer membrane of cells expressing the wild type receptor gives rise to competition between the wild type and variant receptors for available SP. The variant receptor competes with wild type receptor for binding of available SP and decreases the amount of SP available for the wild type receptor. As the NK-1v receptor is unable to transduce a signal upon SP binding, the result is a down-regulation of the action of the wild type receptor.

In the treatment of lung tissue, researchers at Stanford University Medical Center, California have initiated a trial of gene therapy for cystic fibrosis in which the active material is delivered to the lungs by aerosol. The active material consists of a version of the cystic fibrosis trans-membrane conductance regulator gene packed into an adeno-associated virus (AAV) shell. A similar route can be adopted for delivering the hNK-1v receptor to the lungs of patients where the activation of native hNK-1 receptors by receptor ligands requires to be down-regulated. A strategy for down-regulating the action between SP and human NK-1v receptor in lung tissue can be adopted which is similar to the strategy proposed for gene therapy in cystic fibrosis. The same technique of packaging genes into AAV and infection of the lung tissue with aerosol AAV can be used to introduce hNK-1v. Samulski et al (University of North Carolina, Chapel Hill, N.C. 27599, USA) market a vector which can be used to package human NK-1v receptor gene into AAV.

Alternatively, a plasmid containing the human NK-1v receptor (pCMVNK-1v) may be used directly in gene therapy. The plasmid may be prepared as a lipid:DNA complex and administered to the lungs as an aerosol, see Pillai et al., Pharm-Res 15(11): 1743-7 (1998), McDonald et al., Pharm-Res 1998 15(5): 671-9(1998). This strategy can also be used with an NK-1 receptor promoter. A pre-requisite for the use of gene therapy for humans is the ability to make sufficient pharmaceutical grade plasmid DNA. This problem has been addressed by Prazeres et al in TIBTECH 17: 169-74 (1999).

Both rAAV and lipid:DNA complex may be administered to the lungs via nebulisers, e.g. airjet nebulisers. Methods for administration may generally follow the teachings of McDonald et al, Pharm Res 15(5): 671-9 (1998); Yonemitsu et al, Gene Therapy 4(7): 631-8 (1997); McDonald et al, Human Gene Therapy, 8(4): 411-22 (1997); Bellon et al, Human Gene Therapy 8(1): 15-25 (1997); and Niven, Critical Review of Therapeutic Drug Carrier Systems 12(2-3): 151-231 (1995).

Both viral and plasmid therapeutic compositions can also be delivered to other target sites such as joints and nervous tissue by infusion and injection.

### EXAMPLES

Embodiments of the invention will now be described in the following Examples

### Example 1

### Preparation of a Modified Human NK-1 Receptor (NK-1v)

The starting material used was a plasmid pRc/CMV (Invitrogen Co) containing a cDNA clone encoding the human NK-1 receptor. The clone encoded 407 amino acids and was flanked by a Nco I site at the 5′ end (around the ATG start codon) and a Xba I site at the 3′ end following the stop codon. Human NK-1v receptor was made from the above plasmid using a PCR-based strategy that mutated the DRY motif (Asp¹²⁹, Arg¹³⁰, Tyr¹³¹) at the end of third transmembrane helix to GGA.

Portions of the cDNA clone in the above plasmid were amplified in two separate PCR reactions using Pfu DNA polymerase (Stratagene), 20 cycles of PCR using 1 min at 94° C., 1 min at 55° C., 4 min at 72° C., followed by 10 mins at 72° C., 50 pmol of each primer, 50 ng of plasmid DNA, 100 μl reaction volume.

The first PCR reaction used the following primers to produce a predicted product of 473 bp:

(SEQ ID N^{o}7)

5′ primer, sense-A:
5′-AAC TAG AGA ACC CAC TGC TTA-3′

(SEQ ID N^{o}8)

3′ primer, anti-sense-A:
5′-GCC ATA GCG CCG CCA AAA GCC ACA GCC GT-3′

The second PCR reaction used the following primers to produce a predicted product of 888 bp:

(SEQ ID N^{o}9)

5′ primer, sense-B:
5′-TTT GGC GGC GCT ATG GCT ATC ATA CAT CC-3′

(SEQ ID N^{o}10)

3′ primer, anti-sense-B:
5′-AGC TCT AGC ATT TAG GTG ACA-3′.

Primers anti-sense-A and sense-B contained 17 bases of overlapping complementary sequence at the 5′ ends of each to allow annealing of the two products to form the full length mutated receptor. All the above primers were custom synthesized by Perkin-Elmer.

The two PCR products were gel-purified (QIAEX, Qiagen) and 50 ng of each purified product was added to a 100 μl Pfu PCR reaction and three rounds of PCR performed without primers to allow the two overlapping regions of the PCR products to anneal and extend. 50 ng of the flanking primers (sense-A and anti-sense-B) were added and 20 cycles of conventional PCR performed. The resulting full-length product was purified, cloned into pBluescript (Stratagene) and fully sequenced to confirm the mutations had been successfully made.

The clone was modified by site-directed mutagenesis using a Clontech Transformer kit to remove an internal Nco I restriction site using the oligonucleotide sense-C also from Perkin-Elmer.

| | |
|---|---|
| Sense C: | |
| 5′-CGC GGA GGC TTC TAT GGC TGC AT-3′ | (SEQ ID N^{o}11) |

The resulting cDNA was excised from the parental vector, spliced into a mammalian expression vector, pCMV3.1 (Invitrogen). This cDNA was in an orientation which allowed expression of the hNK-1vR polypeptide. Stock plasmid was prepared by amplification in E. coli and the plasmid DNA subsequently purified using a Qiagen endonuclease free DNA preparation kit, and the fidelity of the construct confirmed by DNA sequencing. The nucleic acid sequence of the cDNA is SEQ ID N^{o}6 (FIG. 2) and the translated protein sequence is SEQ ID N^{o}5 (FIG. 1). Similarly control (negative control) constructs were prepared whereby wild type and variant HNK-1R cDNA was spliced into the vector in an opposite configuration to that described above. A further control (positive control) was prepared by ligation of the parental wild type hNK-1R cDNA into the vector in a manner which would allow hNK-1R polypeptide to be expressed.

### Example 2

### COS-7 Cells that Transiently Express Human NK-1v Receptor

COS-7 cells were grown in DMEM culture medium supplemented with 10% foetal calf serum and 2mM glutamine and maintained under an atmosphere of 5% CO₂. Cells were passaged at approximately 70% confluence by reseeding to a concentration of approximately 10% confluence per 175 cm² flask. COS-7 cells were harvested and prepared for electroporation in Equibio electroporation buffer. Cells (5×10⁶ cells) were electroporated at room temperature in a 4 mm gap cuvette in a final volume of 800 μl containing 30 μg of transforming plasmid DNA, with 250 volts, 1500 μF at infinite resistance using an Equibio EasyJect plus electroporator. Transformed COS-7 cells were cultured for 2-3 days in a 175cm² flask prior to assay.

Alternatively cells (approximately 5-50 ul of transfected cells) were cultured in 6 well culture dishes containing 22mm diameter cover slips. These cover slips coated in cells were used for imaging experiments designed to investigate changes in the concentration of intracellular free calcium ([Ca²⁺]i). Cover slips were prepared by first immersing the cover slips in approximately 70% ethanol and then quickly passing through a gas flame to sterilize. Cover slips were then allowed to air dry in a culture hood prior to placing in the bottom of a culture well of a standard six well culture dish. Cells were then cultured as described above.

### Example 3

### COS-7 Cell Membranes Containing Human NK-1v Receptors

The transfected COS-7 cells of Example 2 were harvested by treatment with Versene. (Gibco BRL). Versene was used, as it allows detachment of the cells from the flask without substantial perturbation to cell membrane proteins, then, the cells were washed once by re-suspending in assay buffer (50 mM Tris HCl pH 7.4, 3 mM MnCl₂, 0.02% BSA, 40 μg/ml bacitracin, 2 μg/ml chymostatin, 2 μM phosphoramidon, 4 μg/ml leupeptin) and centrifuged at 1000 g for 5 min. Cells were re-suspended in 5 ml of the assay buffer and a cell count performed prior to lysing cells, using a Brinkman polytron at setting 6 for 15 seconds. The homogenate was centrifuged at 20000 g for 10 minutes. Membrane pellets were re-suspended in assay buffer and stored frozen as 0.5-1 ml aliquots until required for use.

### Example 4

### Ligand Binding to Receptors in Cell Membranes

COS-7 cells were transiently transfected with pCMV3.1 (Invitrogen) containing either parental wild type human NK-1 receptor cDNA or the variant human NK-1 receptor cDNA of Example 1, and cell membranes were prepared 2-3 days post-transfection using the procedure of Example 3. Radioligand binding studies were performed using [¹²⁵I]BH substance P to label NK-1 receptors.

Non-specific binding was defined by the NK-1 receptor-selective agonist [Sar⁹, Met(O₂)¹¹]substance P (Bachem) at a final concentration of 1 μM. On the day when they were required, the membrane suspensions were thawed and diluted as appropriate with assay buffer and incubated with varying concentrations of [¹²⁵I]Bolton-Hunter Substance P (0.05-3 nM, from Amersham Life Sciences) for 50 minutes at 21° C. Saturation analyses were performed to determine the affinity constants and maximum binding capacity for each receptor, by incubating membranes with increasing concentrations of the radioligand, in the presence and absence of [Sar⁹, Met(O₂)¹¹]substance P. Reactions were terminated by rapid filtration under vacuum, onto GFC filters pre-soaked with 0.2% polyethylenimine.

[¹²⁵I]BH substance P bound with high affinity to both wild type and mutant receptors (Kd values of 2.218±1.107 nM (n=3) and 1.446 nM (n=2) respectively). Unpredictably, there appeared to be no significant difference between the dissociation constant and the maximum binding capacity of the wild type and variant NK-1 receptors, indicating that the mutation does not affect agonist binding at the NK-1 receptor. The maximum binding capacity can be directly compared, as shown in Table 1:

TABLE 1

| | Wild Type | Variant |
|---|---|---|
| | HNK-1 (n = 3) | hNK-1 (n = 2) |
| Kd (nM) | 2.218 + − 1.1107 | 1.446 |
| Bmax | 2.201 | 3.000 |

### Example 5

### Analysis of Intra-Cell Receptor Coupling in COS-7 Cells by Changes in Intra-Cellular Free Calcium Concentration

Coverslips containing cells were prepared and maintained as described. Cultured cells were washed twice in a Krebs-Hepes extra-cellular medium buffer (EM, composition in mM: NaCl 118, KCl 4.7, MgSO₄ 1.2, CaCl₂ 1.2, KH₂PO₄ 1.2, Hepes 10, glucose 11 and BSA 0.1%, (pH 7.2 at 20° C., (Rossant, et al Endocrinology, 140, 1525-1536), and then loaded with Fura-2 (Rossant, et al Endocrinology, 140, 1525-1536) by incubation for 3 h at 20° C. with EM containing Fura-2-AM (2 μM, Molecular Probes). This procedure enables the cells to load with Fura-2-AM, which becomes hydrolysed to the free acid form once inside the intact cells. After loading, coverslips were mounted into imaging chambers and perfused with EM to remove extra-cellular Fura-2-AM and to allow hydrolysis of intracellular Fura-2-AM to occur. Measurements of changes in the free [Ca²⁺]ᵢ in individual cells were made from the fluorescence ratio (excitations 340 nm/380 nm, emission >510 nm) using a Spectral Wizard monochromator, cooled integrating CCD camera and a dedicated suite of software (Merlin, Life Sciences Resources, Cambridge, UK). Data are expressed as ratio units 340/380.

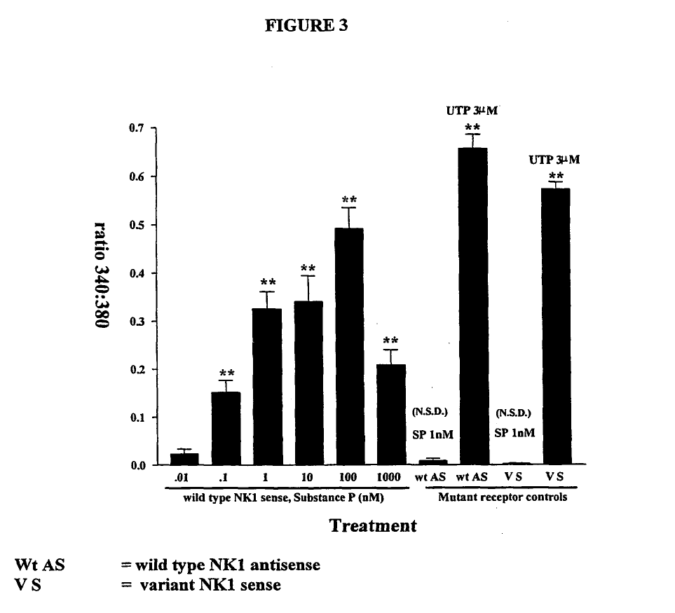

Genes encoding wild type sense, wild type anti-sense and variant NK-1 receptors described above were transiently transfected into COS-7 (monkey kidney) cell lines. Two to three days post-transfection, cells were loaded with FURA-2-AM dye (whose fluorescence is [Ca²⁺]ᵢ dependent) and challenged with substance P. The [Ca2+]ᵢ was followed by monitoring the change in light emitted (>510 nm) when excitation light of either 340 nm or 380 nm was sequentially used to illuminate the loaded cells and expressed as a ratio (340 nm:380 nm). Cells challenged with UTP (which increases [Ca²⁺]ᵢ in non-transfected cells) caused a [Ca²⁺]ᵢ response indicating the presence of a functional G-protein Ca²⁺ linked pathway. Results plotted in FIG. 3 indicate that cells expressing wild type NK-1 receptor responded to substance P over a 0.01-1000 nM range. Cells expressing wild type anti-sense and variant NK-1 receptor failed to invoke a Ca²⁺ response with substance P challenge of 1 nM.

The effect of treatment with varying concentrations of substance-P or with UTP (3μM) for a 1 min time period on the average change in 340:380 ratio levels of COS 7 cultures expressing either wild type or modified NK-1 receptors (FIG. 3, n>50 cells for each treatment group (or on the percentage of COS cells demonstrating a measurable response (Xb) is shown in FIG. 3).

1. A mutant tachykinin receptor in which the three amino acids of the DRY sequence that occurs adjacent to the junction of the TM3 domain with 5 intracellular loop 2 are replaced with amino acids whose side chains are neither lipophilic nor contain charged groups, said receptor exhibiting similar ligand binding characteristics to the wild type receptor but exhibiting substantially no intra-cellular coupling of the receptor to the G-protein, whereby there is substantially no transduction of ligand binding 10 signals to the cell; or
a fragment of said receptor containing said modified DRY sequence; or
an isolated protein or polypeptide containing an amino acid sequence at least 80% identical to the above sequence; or
a variant thereof with sequential amino acid deletions from either the C terminus or the N-terminus; or
an allelic variant, heterospecific homologue or biologically active proteolytic or other fragment thereof containing said modified DRY sequence.

2. A non-human mammalian receptor according to claim 1.

3. A rat or mouse receptor according to claim 1.

4. A human receptor according to claim 1.

5. A mutant NIL-1 receptor according to claim 1.

6. A mutant NK-2 receptor according to claim 1.

7. A mutant NK-3 receptor according to claim 1.

8. A receptor according to claim 1, wherein the replacement amino acids are selected from G and A.

9. The receptor of claim 8, wherein DRY is replaced by GGA.

10. A tachykinin receptor of SEQ ID N^{o}5, or a receptor having at least 80% amino acid identity with the receptor of SEQ ID N^{o}5, and that is capable of binding to substance P but is substantially incapable of initiating its endogenous signal, or a fragment of said receptor.

11. An isolated cell membrane incorporating a tachykinin receptor as defined in claim 1.

12. Any of the following:
(a) an isolated nucleic acid molecule comprising a polynucleotide that encodes a tachykinin receptor as claimed in claim 1;
(b) an isolated nucleic acid molecule comprising a sequence that is hybridizable to the above sequence;
(c) a gene which is the result of extending the above sequence or any sequence that is hybridizable to the above sequence;
(d) a sequence or gene that is functionally equivalent to the above sequence or to a gene that is an extension of the above sequence, i.e. that is not identical to the sequence or gene referred to but functions biologically as equivalent to the sequence or gene referred to, including any allelic variants and heterospecific mammalian homologues, including artificial or recombinant sequences created from cDNA or genomic DNA;
(e) a recombinant vector comprising the above gene sequence; and
(f) a host cell transformed with the vector.

13. Any of the following:
(a) an isolated nucleic acid molecule having the nucleotide sequence of SEQ ID N^{o}6;
(b) an isolated nucleic acid molecule comprising a sequence that is hybridizable to the above sequence;
(c) a gene which is the result of extending the above sequence or any sequence that is hybridizable to the above sequence;
(d) a sequence or gene that is functionally equivalent to the above sequence or to a gene that is an extension of the above sequence, i.e. that is not identical to the sequence or gene referred to but functions biologically as equivalent to the sequence or gene referred to, including any allelic variants and heterospecific mammalian homologues, including artificial or recombinant sequences created from cDNA or genomic DNA;
(e) a recombinant vector comprising the above gene sequence; and
(f) a host cell transformed with the vector.

14. A method for producing a receptor protein having an amino acid sequenceas defined in claim 1, which method comprises the steps of:
(a) inserting said nucleic acid sequence into an appropriate vector;
(b) culturing, in an a culture medium, a host cell previously transformed or transfected with the recombinant vector of step (a);
(c) harvesting cells containing the receptor protein obtained from step (b); and
(d) separating or purifying, from said culture medium or from said host cell, the thus-produced receptor protein.

15. A pharmaceutical composition comprising an effective amount of a modified tachykinin ligand as defined in claim 1 or a nucleic acid sequence encoding said ligand and a pharmaceutically and pharmacologically acceptable carrier.

16. Use of a modified tachykinin receptor as defined in claim 1 in the preparation of a medicament for the treatment or prophylaxis of a condition associated with substance P or other tachykinin (neurokinin) receptor-binding ligand;

17. A method for the treatment or prevention of a condition associated with over-expression or inappropriate expression of an endogenous tachykinin ligand, which method comprises administration to a patient in need thereof of a non-toxic, effective amount of such a modified tachykinin ligand as defined in claim 1.

18. A method for screening for therapeutically active compounds, said method comprising the following steps:
(a) providing a cell line expressing a modified tachykinin receptor as defined in claim 1;
(b) adding test sample to a solution containing labeled tachykinin ligand and the cell line from step (a);
(c) incubating the cell line, test sample and labeled ligand mixture from step (b) to allow binding of said ligand and test sample to the modified tachykinin receptor;
(d) optionally, separating the non-bound labeled ligand from the labeled ligand bound to the modified tachykinin receptor; and, if desired,
(e) measuring the amount of labeled ligand that is bound to the modified tachykinin receptor.

19. Use of a modified tachykinin receptor as defined in claim 1 as a substitute in an assay to identify and/or evaluate entities that bind to the wild type tachykinin receptor.

20. Use of a modified tachykinin receptor as defined in claim 1 as a substitute in an assay in order to determine the concentration of ligand in body fluids in patients with arthritis, pain, migraine, anxiety, schizophrenia, asthma, rheumatoid arthritis, and in gastrointestinal disorders and diseases of the GI tract.

21. An assay procedure comprising the following steps:
(a) providing a cell line is provided that expresses a modified tachykinin receptor as defined in claim 1;
(b) labeling the cell line;
(c) adding the test sample and labeled cells to a matrix binding SP or other ligand;
(d) incubating the labeled cells, test sample and matrix-bound SP or other ligand to allow binding of SP or other ligand and test sample to the expressed modified tachykinin receptor;
(e) separating the labelled non-bound cells from the SP or other ligand bound cells; and, if desired,
(f) measuring the amount of labelled cells containing the modified tachykinin receptor that has bound to SF or other ligand.

22. Use of a modified tachykinin receptor as defined in claim 1 in protein therapy to reduce the effects of an excess of or inappropriately produced endogenous ligand.

23. A method for treatment of a patient in need thereof, which comprises administering to said patient a composition in the form of an aerosol that comprises a modified tachykinin receptor as defined in claim 1.

24. A method for gene therapy treatment of a patient in need thereof, which comprises administering to said patient a nucleic acid sequence, virus or plasmid encoding a modified tachykinin receptor as defined in claim 1.


SEQUENCE LISTING (18 sequences)

SEQ ID NO: 1 - protein, 10 aa, Unknown
  Description of Unknown Organism Mammalian
RPKPQFFGLM

SEQ ID NO: 2 - protein, 10 aa, Unknown
  Description of Unknown Organism Mammalian
HKTDSFVGLM

SEQ ID NO: 3 - protein, 10 aa, Unknown
  Description of Unknown Organism  Mammalian
DMHDFFVGLM

SEQ ID NO: 4 - protein, 11 aa, Artificial Sequence
  Description of Artificial Sequence Conserved sequence
DRYXXVPXXPX

SEQ ID NO: 5 - protein, 407 aa, Artificial Sequence
  Description of Artificial Sequence Modified human NK-1 receptor
MDNVLPVDSDLSPNISTNTSEPNQFVQPAWQIVLWAAAYTVIVVTSVVGNVVVMWIILAHKRMRTVTNYFLVNLAFAEASMAAFNTVVNFTYAVHNEWYYGLFYCKFHNFFPIAAVFASIYSMTAVAFGGAMAIIHPLQPRLSATATKVVICVIWVLALLLAFPQGYYSTTETMPSRVVCMIEWPEHPNKIYEKVYHICVTVLIYFLPLLVIGYAYTVVGITLWASEIPGDSSDRYHEQVSAKRKVVKMMIVVVCTFAICWLPFHIFFLLPYINPDLYLKKFIQQVYLAIMWLAMSSTMYNPIIYCCLNDRFRLGFKHAFRCCPFISAGDYEGLEMKSTRYLQTQGSVYKVSRLETTISTVVGAHEEEPEDGPKATPSSLDLTSNCSSRSDSKTMTESFSFSSNVLS

SEQ ID NO: 6 - DNA, 1224 bp, Artificial Sequence
  Description of Artificial Sequence  Consensus cDNA encoding a modified human NK-1 receptor
atggataacgtcctcccggtggactcagacctctccccaaacatctccactaacacctcggaacccaatcagttcgtgcaaccagcctggcaaattgtcctttgggcagctgcctacacggtcattgtggtgacctctgtggtgggcaacgtggtagtgatgtggatcatcttagcccacaaaagaatgaggacagtgacgaactattttctggtgaacctggccttcgcggaggcttctatggctgcattcaatacagtggtgaacttcacctatgctgtccacaacgaatggtactacggcctgttctactgcaagttccacaacttcttccccatcgccgctgtcttcgccagtatctactccatgacggctgtggcttttggcggcgctatggctatcatacatcccctccagccccggctgtcagccacagccaccaaagtggtcatctgtgtcatctgggtcctggctctcctgctggccttcccccagggctactactcaaccacagagaccatgcccagcagagtcgtgtgcatgatcgaatggccagagcatccgaacaagatttatgagaaagtgtaccacatctgtgtgactgtgctgatctacttcctccccctgctggtgattggctatgcatacaccgtagtgggaatcacactatgggccagtgagatccccggggactcctctgaccgctaccacgagcaagtctctgccaagcgcaaggtggtcaaaatgatgattgtcgtggtgtgcaccttcgccatctgctggctgcccttccacatcttcttcctcctgccctacatcaacccagatctctacctgaagaagtttatccagcaggtctacctggccatcatgtggctggccatgagctccaccatgtacaaccccatcatctactgctgcctcaatgacaggttccgtctgggcttcaagcatgccttccggtgctgccccttcatcagcgccggcgactatgaggggctggaaatgaaatccacccggtatctccagacccagggcagtgtgtacaaagtcagccgcctggagaccaccatctccacagtggtgggggcccacgaggaggagccagaggacggccccaaggccacaccctcgtccctggacctgacctccaactgctcttcacgaagtgactccaagaccatgacagagagcttcagcttctcctccaatgtgctctcctag

SEQ ID NO: 7 - DNA, 21 bp, Artificial Sequence
  Description of Artificial Sequence Primer
aactagagaacccactgctta

SEQ ID NO: 8 - DNA, 29 bp, Artificial Sequence
  Description of Artificial Sequence Primer
gccatagcgccgccaaaagccacagccgt

SEQ ID NO: 9 - DNA, 29 bp, Artificial Sequence
  Description of Aritificial Sequence Primer
tttggcggcgctatggctatcatacatcc

SEQ ID NO: 10 - DNA, 21 bp, Artificial Sequence
  Description of Artificial Sequence Primer
agctctagcatttaggtgaca

SEQ ID NO: 11 - DNA, 23 bp, Artificial Sequence
  Description of Artificial Sequence Oligonucleotide
cgcggaggcttctatggctgcat

SEQ ID NO: 12 - protein, 5 aa, Artificial Sequence
  Description of Artificial Sequence  Shared C-terminal sequence
FXGLM

SEQ ID NO: 13 - protein, 20 aa, Homo sapiens
MTAVAFDRYMAIIHPLQPRL

SEQ ID NO: 14 - protein, 20 aa, Rana catesbeiana
MTAIALDRYMAIIHPLKPRL

SEQ ID NO: 15 - protein, 20 aa, Homo sapiens
MTAIAADRYMAIVHPFQPRL

SEQ ID NO: 16 - protein, 20 aa, Homo sapiens
MTAIAIDRYMAIVHPFQPRL

SEQ ID NO: 17 - protein, 20 aa, Homo sapiens
MTAIAVDRYMAIIDPLKPRL

SEQ ID NO: 18 - protein, 407 aa, Homo sapiens
MDNVLPVDSDLSPNISTNTSEPNQFVQPAWQIVLWAAAYTVIVVTSVVGNVVVMWIILAHKRMRTVTNYFLVNLAFAEASMAAFNTVVNFTYAVHNEWYYGLFYCKFHNFFPIAAVFASIYSMTAVAFDRYMAIIHPLQPRLSATATKVVICVIWVLALLLAFPQGYYSTTETMPSRVVCMIEWPEHPNKIYEKVYHICVTVLIYFLPLLVIGYAYTVVGITIWASEIPGDSSDRYHEQVSAKRKVVKMMIVVVCTFAICWLPFHIFFLLPYINPDLYLKKFIQQVYLAIMWLAMSSTMYNPIIYCCLNDRFRLGFKHAFRCCPFISAGDYEGLEMKSTRYLQTQGSVYKVSRLETTISTVVGAHEEEPEDGPKATPSSLDLTSNCSSRSDSKTMTESFSFSSNVLS